(19) United States
(12) Patent Application Publication
(10) Pub. No.: US 2015/0285326 A1
(43) Pub. Date: Oct. 8, 2015

(54) DAMPER

(71) Applicant: INVENTUS ENGINEERING GmbH, ST. ANTON I.M. (AT)

(72) Inventors: Stefan Battlogg, St.Anton I.M. (AT); Michael Kieber, Schruns (AT); Juergen Poesel, Bludenz (AT)

(73) Assignee: INVENTUS ENGINEERING GmbH, ST. ANTON I.M. (AT)

(21) Appl. No.: 14/425,178

(22) PCT Filed: Aug. 27, 2013
(86) PCT No.: PCT/EP2013/002574

§ 371 Date: May 15, 2015

(30) Foreign Application Priority Data
Aug. 28, 2012 (DE) 102012016948.5

(51) Int. Cl. F16F 9/53; A61F 2/50; B60N 2/50; B60N 2/52; F16F 9/18; B62K 25/04

(52) U.S. Cl. CPC F16F 9/535; F16F 9/187; B62K 25/04; B60N 2/501; B60N 2/522; A61F 2/50; A61F 2002/5004; A61F 2002/5006

(57) ABSTRACT

A damper has a damper chamber divided by a movable piston into first and second chambers, which are connected via a return channel, a controllable throttle valve and a connecting channel. A magnetic field source controlled by an electronic control device applies a magnetic field to a magnetorheological damping medium flowing through the throttle valve. A compensation chamber has a pre-loaded compensation volume connected to the throttle valve and the second chamber. A one-way circuit with one-way valves causes the damping medium to flow in the same direction of circulation when the piston rod plunges into the damper chamber and when it emerges therefrom. A first one-way valve on the piston allows the damping medium to flow from the second into the first chamber. A second one-way valve between the throttle valve and the second chamber allows the damping medium to flow from the throttle valve into the second chamber.

The present invention relates to a damper that is used to damp the movement of a first component relative to a second component, and to a method. The first and the second component can be connected to the damper. In particular other modules or parts will be, or will be able to be connected to the first and the second component. It is possible that such a damper is provided at least partially and in particular practically completely in a tube system, in which a tube, such as an inner tube, and a second tube, which is movable relative to the first tube and in particular can be telescopic, such as an outer tube, are provided as the first and second component.

Various dampers have been known in the prior art. Dampers with field-sensitive fluids have also been known. Magnetorheological fluids (MRFs) are particularly suitable as field-sensitive fluids for use in dampers. Effective damping can be set via a magnetic field.

In the case of magnetorheological fluids, an oil-based damping medium with finely distributed ferromagnetic particles is usually used as damping fluid. For damping, the magnetorheological fluid passes through one or more damping gaps, in which a magnetic field is present. Due to the different damping channels and the various damping valves, dampers are usually constructed in a very complex manner. A particular problem with dampers for muscle-operated modules, components, vehicles and in particular bicycles or prostheses is the overall bulk. A further key factor is the weight, which is of particular importance in particular in the case of prostheses and all the more so in the case of dampers for competition (for example sport, for example Paralympics) and for the demanding amateur field.

Furthermore, it is advantageous if at least individual parts of the damper are dimensioned such that they can be used on other dampers. The assembled dimensions are to be reliably limited in any case. A further key criterion is the possible spring travel.

A particularly important criterion is the basic friction of the damper or the resultant “response behavior”. Dampers with magnetorheological fluid (MR fluid) according to the prior art, such as the MagneRide damper of the BWI Group, have a piston rod compensation by means of dividing piston and pressurized gas or air volumes. This pressure acts on the MRF volume in the damper. This results in an extension force of the piston or piston rod, since the piston surface on the rebound side is reduced by the piston rod surface. In addition the pressure in the damper acts on the seals or sealing lips, for example the piston rod seal, which results in higher friction. The extension force and the higher friction are detrimental to the response behavior of a damper, which has a negative effect particularly in the case of rather small movements (shocks), and these are sometimes transferred in an undamped manner to the body to be damped.

Furthermore, the piston rod compensation volume remains yielding, particularly under action of high pressures. A completely unyielding compression stage cannot be implemented with such dampers.

The object of the present invention is therefore to provide a damper of simple construction, which meets at least some of the above-mentioned requirements and in particular does not have the disadvantages with regard to response behavior.

This object is achieved by a damper having the features of claim 1. Preferred developments of the invention are specified in the dependent claims. Further advantages and features of the invention will emerge from the exemplary embodiment and the general description.

The damper can be used in particular to damp the movement between a first component and a second component movable relative to said first component. The first component and the second component may form a tube system, which can be telescopic. Such a tube system may comprise an inner tube and an outer tube, which is movable relative to said inner tube. The first component and the second component may be part of the damper.

The damper has at least one damper chamber and a controllable throttle valve. The damper chamber is divided by a movable piston connected to a piston rod into a first chamber and into a second chamber. The first chamber is connected to the second chamber via a return channel and the throttle valve.

At least one electronic control device is provided. At least one magnetic field source controlled by the electronic control device is associated with the throttle valve in order to apply a magnetic field to at least part of a magnetorheological damping medium flowing through at least one damping channel and thus provide a damping effect. The magnetic field source can be referred to as a magnet device and preferably comprises at least one electric coil.

At least one compensation chamber with a pre-loaded compensation volume is provided. This can be provided for example by means of dividing piston or diaphragm. The compensation volume is connected to the throttle valve and the second chamber. The throttle valve and the magnetic field source and the compensation chamber are arranged externally and therefore outside the damper chamber. The throttle valve and the electric coil are arranged externally and thus outside the first chamber and the second chamber.

Essentially a one-way circuit is provided for the magnetorheological damping medium, in which circuit at least two one-way valves are provided, such that the damping medium flows around in the same direction of circulation, both when the piston rod plunges into the damper chamber and when the piston rod extends or emerges from the damping chamber. A first of the one-way valves is arranged on the piston and allows a flow of the damping medium from the second chamber into the first chamber.

A second one-way valve is arranged between the throttle valve and the second chamber, thereby allowing the damping medium to flow from the throttle valve into the second chamber.

The damper according to the invention has many advantages. A considerable advantage of the damper according to the invention lies in the simple structure, which is provided by the one-way circuit of the MRF. The magnetorheological damping medium flows in the event of compression (compression stage), when the piston plunges further into the damper chamber, from the second chamber through the one-way valve in the piston into the first chamber. Due to the return channel, the damping medium passes via the throttle valve back into the second chamber where appropriate.

The throttle valve is preferably connected to the second chamber via a connecting channel. The second one-way valve is preferably provided on the connecting channel.

The one-way valves may not only be provided externally on the respective bodies, but may also be arranged at a distance therefrom, provided they are directly connected thereto. The term “on” in the context of the present invention also includes the term “in”, and therefore the one-way valves may also be provided in the piston or in or on a connecting channel between the throttle valve and the second chamber.

The magnetic field source on the whole is preferably arranged outside the first and second chamber and in particular also outside the entire damper chamber, the piston and the piston rod. The magnetic field source is preferably subject to an incident flow always from the same side. The magnetorheological damping medium flows in the piston/cylinder space only in one direction within the one-way circuit.

It is also particularly advantageous that the magnetorheological damping medium is always thoroughly mixed due to the one-way circuit.

The magnetorheological damping medium may comprise at least one magnetorheological fluid. In particular, the damping medium is formed as magnetorheological fluid (MRF). The throttle valve is controllable and comprises at least one magnetic field source or magnet device as field generation device for generating a magnetic field in at least one damping channel of the throttle valve.

The throttle valve is particularly preferably arranged axially adjacently to the damper chamber. It is also particularly preferable that the compensation chamber is arranged axially distanced from the piston. In particular the compensation chamber is arranged axially outside and preferably axially adjacently to the damper chamber and preferably axially beside the second chamber.

The compensation volume is connected to the throttle valve and the second chamber. The compensation volume is in particular connected to the second chamber via the connecting channel. By means of the first one-way valve in particular, it is possible to always situate the compensation chamber and the compensation volume in the low-pressure region, i.e. after the throttle valve. The compensation volume must thus be pre-loaded only with low pressure, and, even with high damping, a rigid system is nevertheless obtained, which does not operate in the compensation volume. In addition, the compensation volume thus causes only a low extension force on the piston, which improves the response behavior significantly.

Such an embodiment has considerable advantages. Both with compression (compression stage) and with extension (rebound stage), at least some of the damping medium flows through the throttle valve. With compression, the piston rod enters further into the damping chamber, such that the damping medium must breach the first one-way valve in the piston in order to pass into the first damper chamber. The path from the second damper chamber outwards via the second one-way valve is closed, since this only allows the damping media to flow through the connecting channel into the second damper chamber. The one-way valve blocks in the opposite direction.

As it plunges in, the piston displaces a volume which is proportional to the cross-sectional area thereof. However, only a volume that is proportional to the cross-sectional area of the piston minus the cross-sectional area of the piston rod is free in the first spring chamber. Therefore, as the piston plunges in, some of the damping medium must flow through the return channel to the throttle valve. A corresponding throttling occurs there. This proportion of the damping medium then enters the compensation chamber.

With extension the piston rod exits from the damper chamber and a volume of the damping medium that is proportional to the cross-sectional area of the damper piston must flow in the second damper chamber. Since the first one-way valve in the piston allows only a flow of the damping fluid from the second damper chamber to the first damper chamber and blocks in the opposite direction, the damping medium must enter the second damper chamber via the connecting channel through the now opening second one-way valve. At the same time, the displaced damping medium exits from the first damper chamber and passes via the return channel to the throttle device. Since in the second damper chamber more volume is required than is displaced in the first damper chamber, a proportion corresponding to the cross section of the piston rods has to be delivered from the compensation chamber. Flows are therefore present in the same direction of circulation both in the return channel and in the connecting channel both with compression and with extension.

This is advantageous since both the rebound stage and the compression stage can be damped via a single throttle valve. This facilitates the construction of such a damper considerably. Weight can also be saved, and the continuous flow leads to good mixing of the magnetorheological fluid.

One advantage of such a solution is that the volume compensation is always arranged in the low-pressure region. This means that the volume compensation as considered in the flow direction is always arranged after the throttle valve or the throttle valves. The pre-load pressure in the compensation volume is preferably below 5 bar. The pre-load pressure may also be just 2 or 3 bar.

In particularly preferred embodiments the damper chamber is arranged and/or connected in such a way that more effectively active piston surface is associated with the rebound stage than with the compression stage. This means that in the rebound stage, with a certain stage, more volume of the damping medium passes through the throttle valve than with the same stage in the compression stage. Here, the expression “effectively active piston surface” is to be understood to mean the relation to the volume flow passing through the throttle valve.

In most constructions the effectively active piston surface is greater in the compression stage. This is true in particular for solutions in which the effectively active throttle valve is arranged in the piston itself.

However, damping conditions that result in greater damping in the rebound stage are often desirable. Such a solution can be achieved here in a constructionally advantageous manner. A rebound stage with a steeper characteristic curve can be made possible in a simple manner.

In preferred embodiments it is possible that the throttle valve is connected to the compensation chamber via a first check valve. Here, the first check valve allows only a flow of the damping medium from the throttle valve into the compensation chamber.

The compensation chamber is preferably connected to the second chamber via a second check valve. Here, the second check valve allows only a flow of the damping medium from the compensation chamber into the second chamber.

At least one of the one-way valves and/or of the check valves is/are particularly preferably settable so as to enable a settable flow resistance in the compression stage and/or the rebound stage. With such an embodiment it is possible that the compensation chamber is connected to the connecting channel via two separate compensation channels. One compensation channel is provided with the first check valve, whereas the second compensation channel is equipped with the second check valve.

In these embodiments it is possible in particular that the check valves can be set externally on the damper, for example in order to change the characteristic curve in the case of mechanical check valves. The check valve is then formed as a settable throttle valve with check device. If, in such an embodiment, a mechanical throttle valve is used, the basic characteristic curve for example is thus set with the throttle valve, whereas for example the basic characteristic curve is adapted via the two settable check valves to the desired characteristic curve for the case of the rebound stage and to the desired characteristic curve for the case of the compression stage.

At least one one-way valve and/or at least one check valve can be adjustable. By way of example, such a valve can be mechanically adjustable in order to change basic settings.

It is possible in particular that the throttle valve is controllable and generates an accordingly controlled magnetic field in the damping channel of the at least one throttle valve. In principle, besides magnetorheological fluids, electrorheological fluids (ERFs) have also been known. However, an MRF is considerably better suited for the intended purpose, since ERFs require high voltages for control. A further disadvantage of ERFs is that it is not possible to induce permanent fields. By contrast, in the case of MRFs, it is possible to set certain throttle states currentlessly using permanent magnets or to utilize the remanence of materials. Here, the magnetic field strength of a permanent magnet is set in a sustainable manner by a short magnetic pulse, for example. The set magnetic field strength is also retained long after the magnetic pulse, without also requiring external energy. These possibilities are not provided in the case of ERFs.

Such an embodiment is particularly advantageous since magnetorheological damping media respond quickly to applied magnetic fields. Here, it is possible that a permanent magnet is used as field generation device. Such a permanent magnet for example can be mechanically modified in terms of the position thereof so as to change the damping force acting in the damping channel. It is also possible that a permanent magnet is used of which the magnetic field is superimposed by the magnetic field of an electric coil depending on the desired requirements. A continuous damping can thus be set by the permanent magnet, said damping for example being attenuated or intensified by the magnetic field of the electric coil as required.

It is also possible that the field generation device comprises what is known here as a remanence magnet, of which the magnetic field strength is set periodically as required or at irregular intervals by a magnetic pulse of an associated electric coil. Such a remanence magnet is set permanently to a certain magnetic field strength by the magnetic pulse of just a few milliseconds duration, for example. When the magnetic field strength of the remanence magnet is to be reduced again, this can be implemented for example via an alternating field that weakens over time. A solution for the fundamental construction of a throttle valve with a remanence magnet can be derived in particular from EP 2 339 203 A2. A preferred construction of a valve working with remanence is preferably oriented to this document.

A particularly flexible control of the damping properties is made possible with an electrically adjustable throttle valve and a magnetorheological fluid. Irrespective of mechanical adjustment possibilities, such a controllable throttle valve provides the possibility of real-time control, in which case a shock is responded to in real time, as the shock becomes stronger, and before it reaches its maximum. This can be ensured here by the reaction speed of a fluid, which for example is a magnetorheological fluid, which can concatenate along the field lines of a magnetic field within the space of a millisecond or slightly longer, and which can thus considerably enlarge the flow resistance transversely thereto.

At least one magnetic field source or magnet device preferably has at least one electric coil, which in particular is mounted outside the first and the second chamber and in which a coil axis is oriented transversely to the flow direction of the magnetorheological damping medium. A particularly high efficacy is thus attained. Such an electric coil can be referred to as a “horizontal” electric coil. The electric coil is provided in particular outside the piston/cylinder space and can even be arranged at right angles to the damping channel in the throttle valve. The electric coil is preferably arranged such that at least a considerable part of the generated magnetic field acts on the damping channel.

In all embodiments at least one control device and/or at least one sensor device is/are preferably provided. The controllable throttle valve can be adjusted with the control device depending on sensor signals. In principle, a wide range of sensors can be provided.

At least one sensor device is particularly preferably provided for identification of at least one control variable.

At least one sensor device is preferably provided in order to detect a measure for a relative speed.

In particular the sensor device is provided to detect a measure for a speed of the piston relative to the damper chamber. However, it is also possible that a sensor device detects a speed of the first and the second component relative to one another. It is also possible that a relative speed of a component is detected for example in a preferred direction (for example in the vertical direction) so as to be able to determine the actual load therefrom. The detection of the acceleration(s) by one or more sensors is also possible. The combination of different sensor types is also possible.

At least one sensor device is particularly preferably provided to detect a direction of the relative movement between the piston and the damper chamber. This is significant for example with the use of magnetorheological fluids, since it is not easily possible to determine merely by the flow of the damping medium within the one-way circuit whether this is flowing damping medium in a compression process or an extension process. In order to solve this problem at least one sensor device for detecting the direction of the relative movement can be provided in simple cases, which sensor device for example comprises at least one deflectable spring plate, which is preferably pre-loaded into a central position by appropriate pre-load devices.

Such a sensor device can be provided for example in the compensation chamber or on a compensation channel, which leads to the compensation chamber. With the compression of the spring plate, which for example serves as a detector, it is possible to detect whether the damping medium is flowing out from the compensation chamber or is flowing into said chamber. Accordingly, with the compression of the detector it is possible to determine whether a compression process or an extension process is present. The detector must be arranged only in a region through which the damping medium flows accordingly in both cases. It is also possible that two separate sensor devices are provided, which separately detect the compression and the extension.

However, it is also possible that a sensor device is provided which detects a measure for a spring travel. Due to the change of the spring travel over time, it is possible to determine whether the damper is in a state of compression or extension. The use of at least one acceleration sensor is also possible, from which or from the data of which it is possible to determine a compression or an extension.

However, it is also possible that a pressure sensor is provided, which measures the pressure difference between the compensation chamber and a reference volume bordering via an aperture. The pressure difference via the aperture opening is then proportional to the compression or extension speed.

In all embodiments it is possible and preferable that at least an end position damping is provided. Such an end position damping may accordingly intensify the damping in an end region in the event of compression or extension so as to prevent a breakthrough at the damper.

With intended use the first chamber is particularly preferably arranged on one side of the second chamber. The throttle valve is then preferably arranged on the other side of the damper chamber. The compensation chamber is in turn preferably arranged adjacently to the throttle valve. With intended use the first chamber is particularly preferably arranged below the second chamber. The throttle valve is preferably arranged above the damper chamber. The compensation chamber is particularly preferably provided above the throttle valve. The compensation chamber can also be provided below or to the side of the throttle valve, which reduces the flow paths.

A reverse construction or a reverse or horizontal use is also possible and preferred. Then, with intended use, the first chamber is arranged above (or for example to the left of) the second chamber. The throttle valve is preferably arranged below (to the right of) the damper chamber. The compensation chamber is particularly preferably provided below (to the right of) the throttle valve. The compensation chamber can also be provided above (to the left of) or to the side of the throttle valve, which reduces the flow paths.

In such embodiments a particularly simple construction is made possible. At the same time, the compensation chamber can be refilled easily, for example with compressed air. The throttle valve arranged above or the compensation chamber arranged thereabove also allows simple refilling or simple exchange of damping medium. Heat can also be dissipated easily.

A considerable advantage of such an embodiment is also that the space provided around a damper can be utilized advantageously.

In certain preferred developments at least the damper chamber and the throttle valve are arranged in a tube system. Here, the first component corresponds to a first tube and the second component corresponds to a second tube, and these tubes in particular can be telescopic.

Whereas in the case of such tube systems only a small diameter is usually available, the length of the tubes can be utilized within said tubes.

Insert devices or a least one insert device is/are particularly preferably provided between the tube system and the damper chamber. Here, the insert device is formed in such a way that the return channel is provided at least in portions at the insert device. The insert device preferably delimits the flow cross section of the return channel. The flow cross-section of the return channel can be considerably reduced by the insert device. The total weight of the damper can thus be considerably reduced, which is of considerable advantage where high demands are placed.

In particular a maximum length, extension or a maximum diameter of a flow cross section of the return channel at the insert device is smaller than a diameter of the tube system. In particular a maximum length or a maximum extension of the flow cross section at the insert device transversely to the flow direction is smaller than a radius and particularly preferably smaller than a half radius of the tube system. In particular the dimensions of the tube system here relate to the outer diameter and particularly preferably the inner diameter of the inner tube.

It is possible that the clearance between the outer peripheral wall of the damper chamber and the inner wall of a tube is used completely as flow channel. With such an embodiment the entire clearance between the outer wall of the damper chamber and the inner wall of the inner tub would be filled here with the damping medium. Due to the significant volume of this clearance, a considerable quantity of damping medium would be provided there, which would rather considerably increase the total weight of the damper. A solution for reducing the weight could lie in reducing the clearance, for example by reducing the inner space or the inner diameter of the inner tube. A smaller clearance would thus be produced, and therefore a lower mass of damping medium would be provided there. However, such a solution would have the disadvantage that the damper would no longer be compatible with conventional dimensions. It would not be possible to use tubes as are currently conventional. This would make the construction of such a damper much more complex.

Alternatively, the outer diameter of the damper chamber could also be enlarged in order to enable a smaller opening in the clearance. With this solution as well a lower mass of damping medium would collect in the clearance, and therefore the weight could be reduced. However, a disadvantage of this solution is that the wall friction as the damping medium flows through would increase rather considerably. It would therefore be difficult to set the required damping properties, since lower damping values would now be virtually impossible to set due to the high flow resistance in the gap.

Even with a reduction of the inner diameter of the inner tube, a corresponding increase of the flow resistance of the damping medium would also be provided.

This means that both a reduction of the diameter of the inner tube as first component and an enlargement of the damper chamber would not provide a satisfactory solution. The surprising solution is then to position at least one insert device in the clearance, said insert device delimiting a precisely defined return channel. Here, the return channel at the insert device preferably has a small peripheral area compared to the cross section thereof. The wall friction at the return channel is thus reduced. The large cross-sectional area compared with the peripheral area allows high flow rates of the dumping medium, without inadmissibly increasing the flow resistance.

The insert device is preferably additionally constructed from such a material and in such a way that a mean density of the insert device that is smaller than a mean density of the damping medium is provided between the tube system and the damper chamber. As a result of such a measure it is ensured that weight can be saved.

In preferred embodiments the mean density of the insert device is less than half the mean density of the damping medium or is at least less than three quarters the density of the damping medium. A considerable weight reduction of the damper can thus be provided, whereas at the same time both high damping rates and low damping rates can be set. The weight can be considerably reduced by sealed cavities in the insert device or by particularly lightweight materials.

In all embodiments it is preferable that the flow of the magnetorheological damping medium can be varied at the throttle valve by means of the magnetic field source or magnet device, and the switched state thereof can then be kept currentless.

In preferred embodiments at least one further throttle valve is provided, for example as a lowering valve with a further field generation device. The further throttle valve can be provided in particular to lower (bend) the damper in the case of prostheses when sitting or to hold said damper in a lowered (bent) position.

The further throttle valve, for example as lowering valve, may comprise an electric coil as field generation device, similarly to the above-described throttle valve. It is also possible that the further throttle valve, similarly to the above-described throttle valve, has at least one remanence magnet and/or at least one permanent magnet as magnet device or field generation device. In all embodiments the further throttle valve is preferably connected in series to the throttle valve.

If, for example, an electric coil is used, a corresponding magnetic field is then only generated with this magnet device when the lowering of the damper is desired, for example when sitting, and when the damper is in the extension state. Then the increased damping ensures a reliable positioning of the damper in the lowered state. At the same time, shocks can also be damped. Later, the lowering valve can be switched off again, and therefore the damper quickly resumes its normal extended position in normal operation (for example walking).

If, for example, only one permanent magnet is selected as magnetic field source for the lowering valve, this will act regularly both in the case of the rebound damping and in the case of the compression damping. A stronger damping behavior is thus produced by the damper following first-time compression.

It is possible that for example a permanent magnet is mechanically movable between a normal position and a further position, such as a lowered position. The permanent magnet may be provided for example on a rotatable device, which surrounds the tube system externally. The permanent magnet can be brought into the desired angular position by means of an adjustment lever, in which position it acts contactlessly on the further throttle valve through the tube system.

In all embodiments it is preferred that at least one one-way valve and/or at least one check valve is/are formed as a shim valve. Such a shim valve may have a stack of different discs, which provide non-linear behavior at the check valve.

At least one one-way valve and/or at least one check valve is/are particularly preferably adjustable. This can be implemented externally, for example.

It is preferable that at least one adjustable valve device is provided, which has remanence properties. An adjustable valve device can consist of a valve or of two or more individual valves connected in series. One of the valves can be formed as a shutoff valve, which in particular purely mechanically, for example as a shim valve, allows the damping medium to pass in just one direction. A further valve or partial valve can be integrated into the shutoff valve of the adjustable valve device and or can be arranged adjacently to the shutoff valve. The further valve can operate on a mechanical and/or electrical and/or magnetorheological basis and can damp the flow through a damping channel of the further valve to a desired extent by generating or applying an adjustable, predetermined or fixed magnetic field. The further valve within the adjustable valve device can be provided on the basis of remanence. Then, an electric coil for generating magnetic pulses is associated with the further valve, with which coil a permanently acting magnetic field in a hard-magnetic or soft-magnetic material is modified or adjusted.

It is also possible that the adjustable valve device has at least one permanent magnet and/or at least one electric coil for generating or applying a desired magnetic field.

It is also preferable that at least one adjustable valve device comprises a remanence valve or consists of just a remanence valve, which operates on a magnetorheological basis and of which the magnetic field can be adjusted by at least one pulse of an electric coil.

In principle, it is preferred that at least one adjustable valve and/or at least one check valve is embodied as an adjustable valve device.

In the case of the damper according to the invention everything can be constructed in a compact manner. All components can be nested inside one another and arranged on top of one another. The electronic control device (electronics) and the battery are thus preferably arranged adjacently of the compensation volume, as considered axially, and therefore the compensation volume is arranged between the battery and the damper chamber.

A method according to the invention is used to provide a damper with structurally induced low-water basic friction and at least a rigid compression stage, which is maintained even with high damping, in particular because it does not operate in the compensation volume. To this end the valves are preferably connected such that the compensation volume as considered in the flow direction is always arranged after the throttle valve. In particular the rebound stage is also rigid, the rigidity being maintained even with a high damping. This is preferably achieved in that the valves in particular are connected such that the compensation volume is always arranged after the throttle valve as considered in the flow direction, even in the case of the rebound stage.

Further advantages and features of the present invention will emerge from the description of the exemplary embodiments, which are explained hereinafter with reference to the accompanying figures.

In the figures:

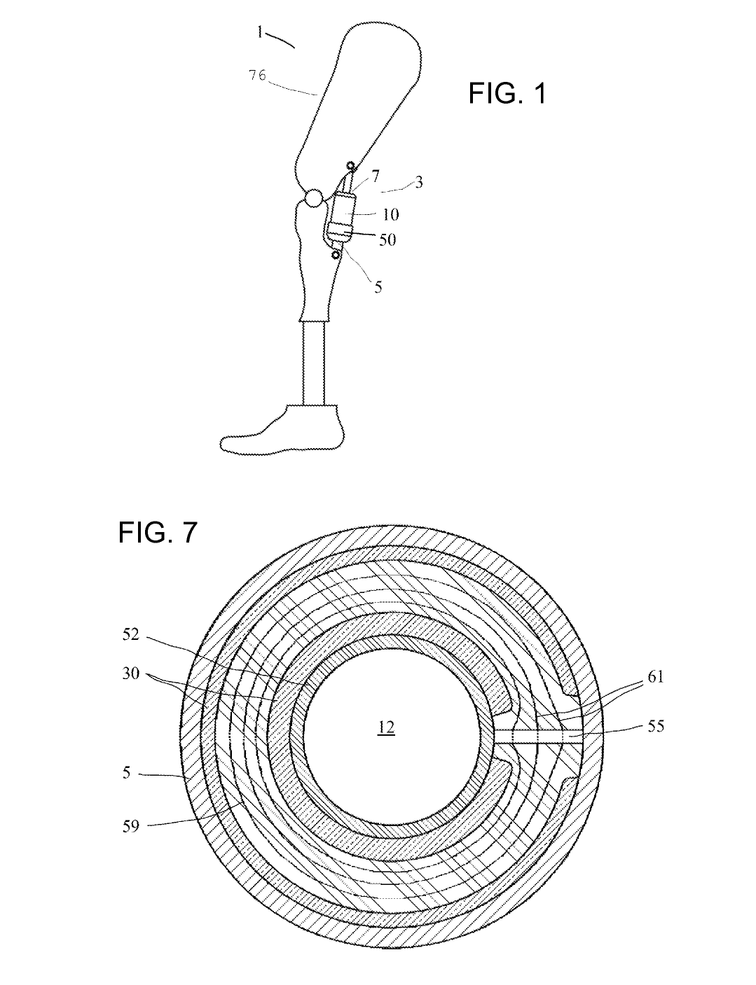

FIG. 1 shows a front view of a damper according to the invention;

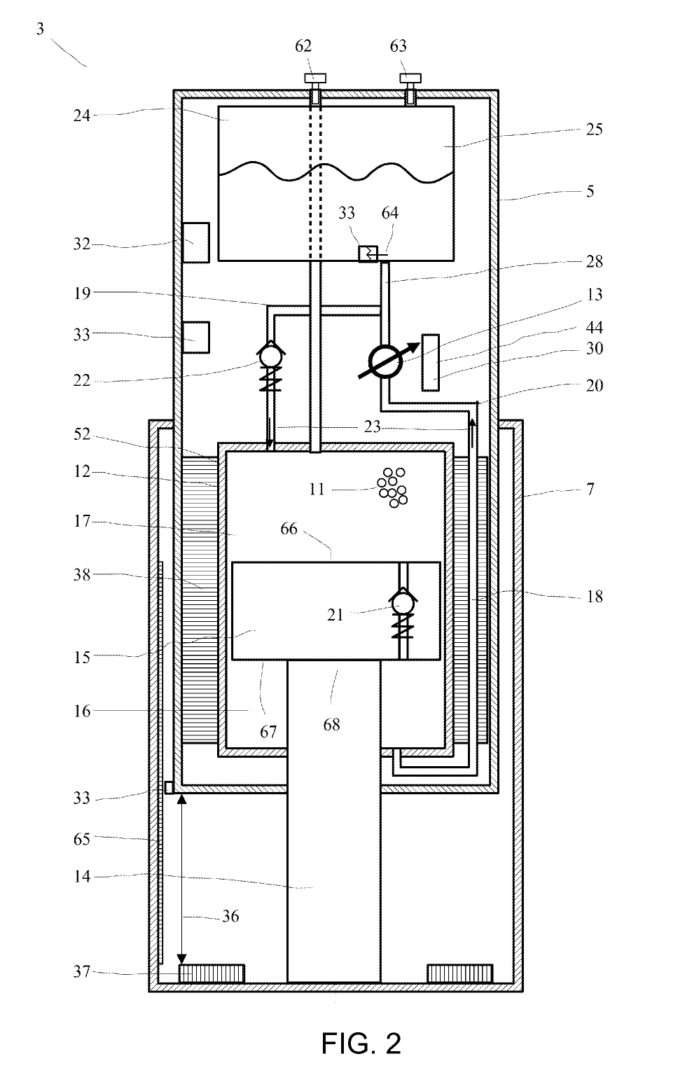

FIG. 2 shows a tube system with a damper device for the damper according to FIG. 1 in a first embodiment;

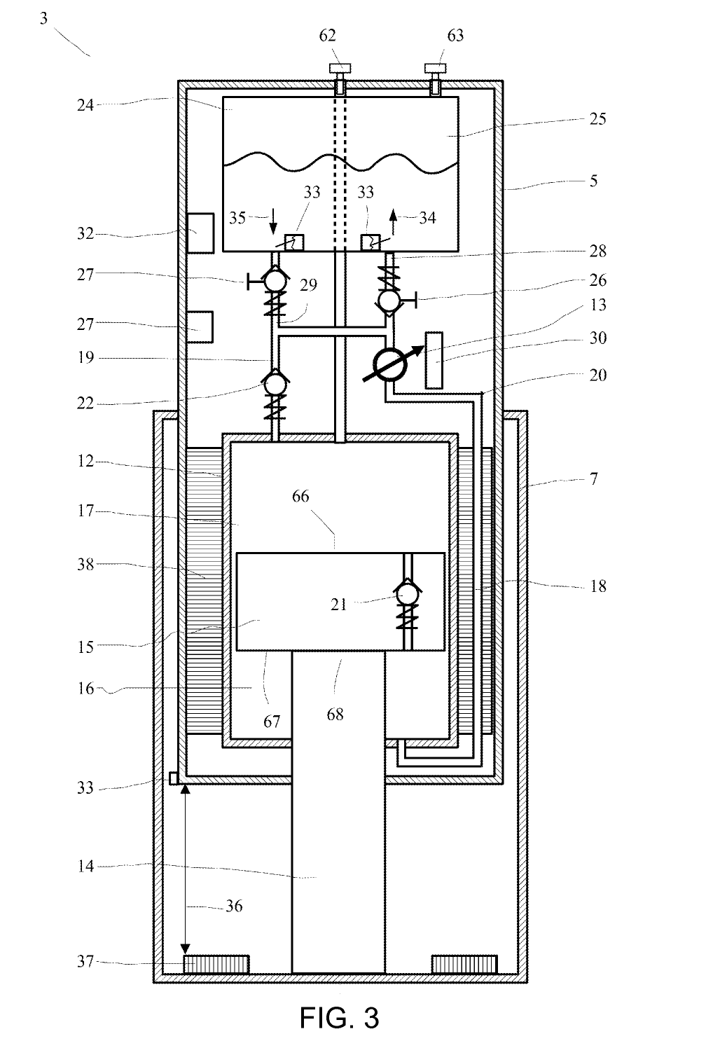

FIG. 3 shows a tube system with a damper device for the damper according to FIG. 1 in a second embodiment;

FIG. 4 shows a tube system with a damper device for the damper according to FIG. 1 in a third embodiment;

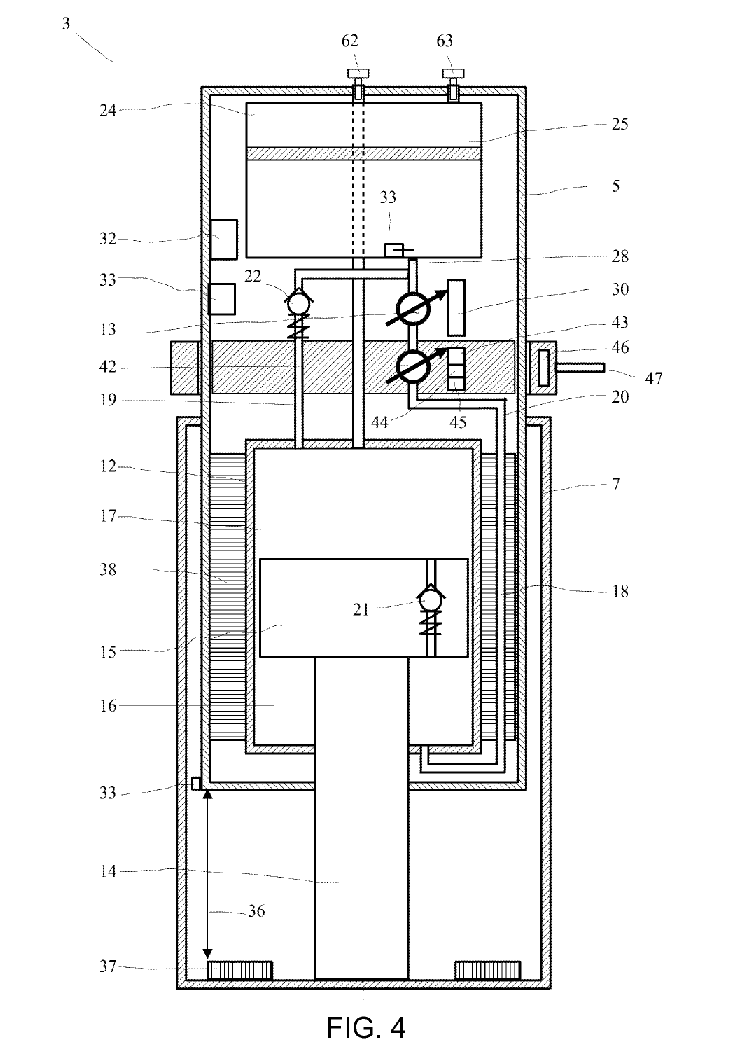

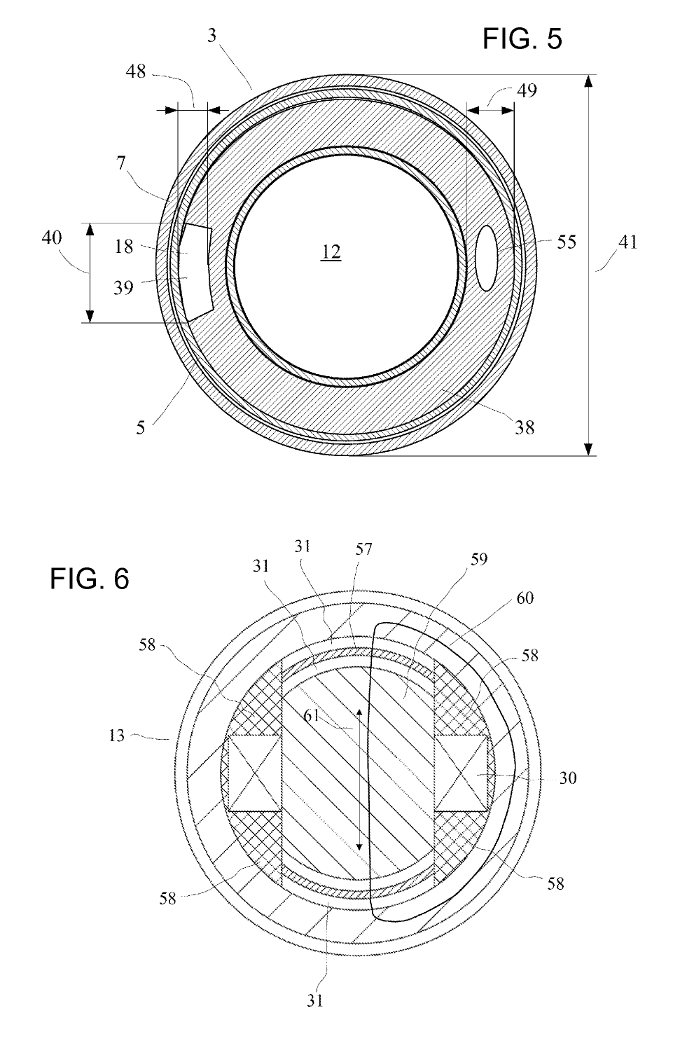

FIG. 5 shows a schematic cross section through a tube system of a damper according to FIGS. 2 to 4;

FIG. 6 shows a schematic cross section through a throttle valve, as can be used in a preceding exemplary embodiment; and

FIG. 7 shows a schematic cross section through a tube system with a throttle valve as can be used in a preceding exemplary embodiment.

FIG. 1 shows, as a possible application of this invention, the use thereof as a damper in a leg prosthesis 76. In this application a low basic friction, a lowering and an unyielding compression stage are necessary, since a prosthesis without this could lead to stumbling and/or the wearing comfort could be significantly reduced.

FIG. 2 shows a schematic cross-sectional illustration of the tube system 3 of the damper from FIG. 1.

A first component 5 as a first tube (inner tube) is connected to an end of the damper 1 or the damper device 10. The second component 7 as second tube is connected to the other end of the damper device or to the piston rod. The two tubes 5 and 7 are provided such that they can be telescopic and can slide here over one another. However, a rotary movement of two components 5 and 7 relative to one another is also possible. The damper 1, besides the actual damper device 10, may also comprise the first component 5 and the second component 7.

The damper device 10 comprises a damper chamber 12, which is divided by a piston 15 into a first chamber 16 and a second chamber 17.

The piston 15 is provided with a piston rod 14, which extends through the first chamber 16 and out from the tube 5. The other end of the piston rod is connected to the lower end of the tube 7 as outer tube 7. The throttle valve 13 is arranged above the damp chamber 12 and is electrically settable. A field generation device 30 is associated with the throttle valve 13 and is used to generate a magnetic field. A magnetorheological fluid is used as damping media 11.

A first one-way valve 21 is provided in the piston 15, which is otherwise embodied as a pump piston. The one-way valve 21 can be embodied for example as a shim valve and allows only the flow of the magnetorheological damping medium 11 from the second chamber 17 through the piston 15 into the first chamber 16 when the pressure within the second chamber 17 is greater than within the first chamber 16. The one-way valve 21 blocks in the opposite direction.

The return channel 18 starts at the end of the first chamber 16 which here is the lower end, and the damping medium 11 can flow through said return channel from the first chamber 16 to the throttle valve 13. The damping medium 11 flowing in the direction of circulation 23 flows through the throttle valve 13, where it is damped in accordance with the settings of the magnetic field source 30 or magnet device 30.

The connecting channel 19 adjoining the throttle valve 13 leads to the second one-way valve 22, which opens in the direction of circulation 23 when the pressure in the connecting channel 19 is greater than the pressure in the second chamber 17. Here, the compensation channel 28 branches off from the connecting channel 19 to the compensation chamber 24, in which a compensation volume 25 is provided. By way of example, the compensation volume 25 may be a flexible bellows or a balloon or the like subject to overpressure and that is resiliently separated from the volume of the compensation chamber 24. The use of a divider piston or a spring for pre-loading is also possible.

The return channel 18 runs through the gap between the damper chamber 12 and the inner peripheral surface of the first tube as first component 5. There, an insert device 38 is located in the gap, which insert device provides a defined cross section for the return channel 18. The volume of the damping medium 11 can thus be considerably reduced, since only the cross section of the return channel 18 at the insert device 38, and no longer the entire gap, is filled with the damping medium 11. The weight of the damper device 10 and of the entire damper 1 can thus be considerably lowered.

A control device 32 is used for control. The control device is a computer that controls, regulates and monitors functions. This may be an open control or a closed regulation, the ACTUAL state measured with a sensor being comparable with a calculated TARGET state (feedback) and the deviation in the closed control circuit then being minimized via the damper. The control device may be equipped with a 32-bit microcontroller in order to process the computing operations required in real time sufficiently quickly and accurately. However, it may also be advantageous to supplement the control device with a field programmable gate array, since these perform digital functions more quickly. The control device has an integrated interface for analog and digital input signals from said sensors and output signals for the damper as well as the fieldbus (for example CAN bus) for communication with other control apparatuses.

The controller 32 is connected to sensors 33, which identify the actual state of the damper and respond thereto accordingly. By way of example, a sensor device 33 may detect the spring travel 36 at short time intervals, such that relative speeds and thus acceleration values also can be determined from the course over time of the signals. The use of acceleration sensors is also possible. The spring travel 36 can be detected via a position identification of the sensor device 33 relative to a measuring device 65.

End position damping means 37 can be provided in order to prevent a striking of the damper.

In operation a shock leads to a compression of the piston 15. Since the damping media cannot escape upwardly via the one-way valve 22 and since the pressure in the second chamber 17 rises, the first one-way valve 21 opens and damping medium 11 flows from the second chamber, through the first one-way valve 21, into the first chamber 16.

Since with compression more damping medium is forced into the second chamber 11 than is available in the first chamber 16, the volume corresponding to the plunging piston rod 14 must flow through the return channel 18 in the direction of circulation 23 to the throttle valve 13, where the damping channel 31 of the throttle valve 13 is exposed to the magnetic field of the field generation device 30. The damping medium is thus damped accordingly.

The damping medium 11 flows a short distance from the throttle valve 13, through the connecting channel 19 and then through the compensation channel 28 into the compensation chamber 24. The inflow of the damping medium 11 can be detected by the detector 64 of the sensor device 33 at the inlet into the compensation chamber 24 or within said chamber. Since the detector plate used as detector 64 bends or twists here in the flow direction, the case of the compression stage can be determined.

With extension, that is to say in the case of the rebound stage, the piston 15 moves downwards in the illustration according to FIG. 2 and a corresponding part of the piston rod 14 exits again from the damper chamber 12. The damping medium 11 located in the first chamber 16 cannot breach the now blocking one-way valve 21 and pass into the second chamber 17, but must flow through the return channel 18 in the same direction of circulation 23 as was the case in the compression stage.

The damping medium 11 flowing through the return channel 18 flows through the throttle valve 13, where it is now exposed to an accordingly adapted magnetic field of the electric coil 44 of the magnetic field source 30.

Since in the case of the rebound stage, due to the piston rod volume, only less damping medium 11 exits from the first chamber 16 compared with the amount required as compensation in the second chamber 17, the second one-way valve 22 opens and damping medium exits from the compensation chamber 24. The damping medium enters the second chamber 17 through the compensation channel 28 and the connecting channel 19. As the damping medium 11 exits from the compensation chamber 24, the detector plate as detector of the sensor device 33 deforms accordingly, such that the case of the rebound stage can be confirmed. A key point here is that, however, in the case of the rebound stage a greater quantity of the magnetorheological damping medium 11 passes through the throttle valve 13 than with the same stroke in the case of the compression stage. This is due to the fact that some of the damping medium 11 is introduced into or removed from the compensation chamber.

The ratio of the gradients of the characteristic curves in the rebound stage and in the compression stage can thus be adapted. It is particularly advantageous for many applications that the rebound stage characteristic curve can be set steeper than the compression stage characteristic curve. The setting can be set structurally via the surface ratios. The piston surface 66 acts in the compression stage. In the rebound stage the piston surface 67 is effective. The difference is given from the piston rod surface 68.

When determining the characteristic curves, it must be ensured however that only a volume proportional to that of the piston rod surface 68 flows within the circuit 20 in the compression stage. The other proportion flows only through the first one-way valve 21. By contrast, in the case of the rebound stage, the ring proportion of the piston flows, that is to say the piston surface 67, which is calculated from the surface 66 minus the surface 68.

Depending on the diameter of the piston rod 68 and the diameter of the piston 15, the flow conditions and therefore the characteristic curve gradients can be varied in the case of the compression stage and rebound stage.

FIG. 3 shows a further exemplary embodiment, the tube system 3 of a damper 1 according to FIG. 1 being illustrated in a likewise highly schematic cross section. In principle, the tube system 3 according to FIG. 3 is structured similarly to the tube system 3 according to FIG. 2. In contrast to the illustration according to FIG. 2, the connecting channel 19 after the throttle valve 13 is divided however into two compensation channels 28 and 29 for exchange with the compensation chamber 24.

In the first compensation channel 28 from the throttle valve 13 to the compensation chamber 24, a first check valve 26 is provided, which allows the damping medium 11 to flow through only from the throttle valve 13 into the compensation chamber 28. A sensor device 33 at the input of the compensation chamber can identify the direction 34 of the relative movement and therefore close in the case of the compression stage.

So that the damping medium 11 can exit from the compensation chamber 24, the second compensation channel 29 is provided, at which a second check valve 27 is arranged. This check valve 29 opens only when the pressure in the compensation chamber 24 is greater than the pressure in the connecting channel 19.

Here, in the exemplary embodiment, the check valves 26 and 27 are settable. It is possible that operating elements are provided externally on the damper, such that the check valves 26 and 27 can be operated where appropriate, for example by the wearer of the prosthesis. To this end, adjustment wheels can be provided accordingly. An electrical remote control is also possible.

In these embodiments the throttle valve 13 is an electrically settable throttle valve, and a magnetorheological fluid is used as damping medium 11.

Although the damper 1 according to FIG. 3 is also operated with a magnetorheological fluid, it may nevertheless be favorable to provide the check valves 25 and 26 in a settable or (pre-) adjustable manner, since an adaptation can thus be made to a basic curve. The throttle valve 13 may then be set depending on the situation.

As already illustrated in FIG. 1, valves 62 and 63 are also provided in FIG. 2 and FIG. 3. The valve 62 can be used for refilling or for exchanging damping medium 11, whereas the valve 63 can be used for example to check the air pressure in the compensation volume 25 of the compensation chamber 24, or for the refilling of compressed air.

FIG. 4 shows another tube system 4 for a damper 1 according to FIG. 1. This tube system 4 is in principle structured similarly to the tube system 4 according to FIGS. 2 and 3.

In contrast to the embodiment according to FIG. 2 the tube system according to FIG. 4 also has a further throttle valve or lowering valve 42, which is arranged in series and here before the throttle valve 13. Here, the damper device 10 is equipped with a magnetorheological fluid as damping medium 11, and therefore the magnetic field sources 30 and 43 are provided for the throttle valve 13 and the lowering valve 42.

The magnetic field source 43 or magnet device 43 according to FIG. 4 may also comprise an electric coil 44, which generates a corresponding magnetic field. It is also possible that for example a remanence magnet 45 is provided, of which the field strength is set to the currently desired value as required or at periodic intervals by magnetic pulses of the electric coil 44. A permanent magnetic field can thus be generated in the remanence magnet 45, which is also available following disconnection of the current required the electric coil 44. The magnetic field strength of the magnetic field source 43 can also be modified as required by a magnetic field of the electric coil 44.

Alternatively or additionally, a permanent magnet 46 can also be provided on an external operating lever, which for example is arranged rotatably about the inner tube 5. By positioning the permanent magnet 46 in such a way that the magnetic field thereof acts on the lowering valve 42 in the desired manner with a magnetic field, a corresponding magnetic field can be generated in the lowering valve 42. By turning away, the magnetic field no longer acts on the lowering valve 42.

In the simplest case only an electric coil 44 is used for field generation. It is then possible in a simple manner to prevent a damper compressed to a certain extent from automatically extending. This is achieved in that an additional magnetic field is always produced at the lowering valve 42 in the case of the rebound stage, which additionally damps the extension. This leads to a permanently lowered damper, which for example is advantageous when sitting (prosthesis). FIG. 5 shows a typical cross section through a tube system according to FIG. 2, 3 or 4. Here, the uncut damper chamber 12 can be seen in the middle. The inner tube 5 of the tube system 3 is illustrated in section radially outwardly. The outer tube 7, which can be telescoped with respect to the inner tube 5, adjoins the latter radially outwardly.

There is a radial distance 49 to the damper device 10 or the damper chamber 12, which distance is filled here practically completely buy an insert device 38. The insert device 38 may be formed in one part, but may also be formed from two or more parts. The insert device 38 extends in the exemplary embodiments substantially over the length of the damper chamber 12, but may also be longer or shorter.

On one side a return channel 18 is provided at the insert device 38. Here, the return channel 18 serves as a return channel for the damping medium 11 over the path from the first chamber 11 via the throttle valve into the compensation chamber 24 or into the second chamber 17. The flow channel 18 for example may have the form illustrated here or other forms, such as round, square or rectangular forms. In principle, any other form is also possible, such as an elliptical form.

It is particularly advantageous if the ratio of the cross-sectional area as flow cross section 39 to the periphery of the return channel 18 is large, such that the flow resistance of the damping medium 11 in the return channel 18 remains relatively low, even with high flow rates. To this end, the ratio of the greatest diameter or the longest extension 40 to the width 48 is relatively small. In particular, the length 40 is smaller than the diameter 41 of the tube system and in particular smaller than the radius of the tube system, preferably also smaller than the half radius of the tube system. On the other hand, the flow cross section 39 is as large as necessary.

On the other side, a similarly formed channel 55 may be provided. It is possible that both channels for example have a rectangular or elliptical cross section. The other channel 55 can be used for example to pass through electrical lines or the like. It is also possible that both channels are used as return channels.

On the whole, the insert device may be solid, and it is also possible that the insert device 38 has hollow regions or hollow chambers, such that the average density of the insert device 38 is reduced. The insert device may consist at least in part of metal and/or plastic.

The average density of the insert device 38 at least in the region of the gap between the tube system 4 and the damper chamber 12 is lower than the density of the damping medium 11 and in particular is at most half as much. A considerable weight proportion can thus be saved. Tests have shown that the weight of the damper device could be reduced by significantly more than 10%. 20% and more may also be possible.

FIG. 6 shows a schematic cross section of a throttle valve 13 illustrated by way of example. A core 59 is provided centrally within the throttle valve 13 illustrated here and is surrounded by a wound electric coil as the field generation device 30. Here, a total of four damping channels 31 are provided, which are separated from one another in twos by a compartment or a compartment-like structure 57. Efficacy is thus increased.

The field lines 61 with applied magnetic field run through the core 59, pass approximately perpendicularly through a damping channel 31, pass through the adjoining compartment 57 and the second damping channel 31, and are guided around the core, here for example in a semi-circle, through the ring 60 made of a magnetically conductive material, as far as the lower region, where two damping channels 31 with intermediate compartment wall 57 are again provided, through which the field lines pass approximately perpendicularly, such that in particular closed field lies 61 are provided. In FIG. 4 only one field line is illustrated by way of representation.

Magnetic insulating materials 58 are provided adjacently to an electric coil 30 in order to shape the magnetic field as desired.

FIG. 7 shows a schematic cross section through a tube system with a throttle valve. The tube system can be used in one of the previously described other embodiments. A cylinder with a wall 52 is arranged in the inner tube 5. The wall 52 delimits the damper chamber 12. A coil as magnetic field source 30 is provided between the cylinder and the inner tube 5. The coil is wound around the ring core 59. The ring core 59 has at one point a slit. This slit forms the damping gap 55, through which the magnetorheological fluid must pass. The damping gap 55 is acted on by the magnetic field controlled by the electronic control device. Some magnetic field lines 61 are illustrated by way of example and pass through the damping gap 55 approximately perpendicularly. This embodiment enables an efficient damper. The damper chamber 12 can be provided internally.

This embodiment according to FIG. 7 with the long thin cylinder coil illustrated here provides considerable advantages. The magnetic voltage can be calculated via the product from number of turns and coil current. The coil will now generate a defined magnetic field in the magnetic circuit, for example a certain magnetic flux or a field of certain magnetic field strength. In accordance with the formula, the coil can also be calculated as an individual turn, in which an accordingly high current flows (specifically the coil current multiplied by the number of turns).

For a magnetic circuit it is essentially irrelevant whether little current circles around the core over numerous thin turns or accordingly more current circles around the core over few turns or even one individual turn, provided the coil current multiplied by the number of turns remains constant.

The losses are given from the current square and the electrical resistance. The individual conductors also cannot be packed arbitrarily densely against one another, and, due to the insulation and the geometric structure, the coil has a (copper) filling factor below 100%. Proceeding from a predefined installation space and identical number of turns, a coil with higher filling factor consequently has more conductive material (=thicker wires), whereby the resistance and thus the power dissipation decrease. Proceeding from the same number of turns and same wire thickness, a higher filling factor means a smaller coil. The mean turn length and thus also the electrical resistance thus generally decreases. A particularly low energy consumption can therefore be attained with the construction illustrated in FIG. 7, which is very important and advantageous in particular in the case of outdoor products.

In all embodiments the magnetic field source or magnet device 30 is preferably arranged on the whole outside the first and second chamber 16, 17 and in particular also outside the entire damper chamber 12, the piston 15 and the piston rod 14. The magnet device 30 is in all cases subject to an incident flow always from the same side. The magnetorheological damping medium 11 flows in the piston/cylinder space only in one direction within the one-way circuit 20.

It is also advantageous that the magnetorheological damping medium 11 is always thoroughly mixed on account of the one-way circuit 20.

The electric coil 44 mounted outside the first and second chamber 16, 17 is arranged in such a way that the generated magnetic field runs at least in part through the damping channel of the throttle valve. In particular, the electric coil 44 is arranged in such a way that an axis of symmetry of the electric coils is oriented transversely to the flow direction of the magnetorheological damping medium.

A damper as previously described is provided and suitable in particular for a seat of a vehicle, in particular such as a passenger car, truck, bus or another utility vehicle or a military vehicle, squad vehicle, a tank, helicopter, a land vehicle or a construction machine.

On the whole, the invention provides an advantageous damper that has very advantageous properties as a result of the use of magnetorheological fluids. A large stroke is enabled, since the superstructures in the tube systems 3 can be made small. In the normal case only a single throttle valve 13 is required in order to damp effectively and differently, both in the case of the compression stage and in the case of the rebound stage.

Due to the use of an insert device, the total weight of the usable damper can be lowered by almost 5% or more, which significantly increases the appeal for example for wearers of a prosthesis.

List of reference signs:

| | |
|---|---|
| 1 | damper |
| 3 | tube system |
| 5 | inner tube |
| 7 | outer tube |
| 10 | damper device |
| 11 | damping medium |
| 12 | damper chamber |
| 13 | throttle valve |
| 14 | piston rod |
| 15 | piston, pump piston |
| 16 | first chamber |
| 17 | second chamber |
| 18 | return channel |
| 19 | connecting channel |
| 20 | one-way circuit |
| 21 | first one-way valve |
| 22 | second one-way valve |
| 23 | direction of |
| | circulation |
| 24 | compensation chamber |
| 25 | compensation volume |
| 26 | first check valve |
| 27 | second check valve |
| 28 | compensation channel |
| 29 | second compensation |
| | channel |
| 30 | magnetic field source, |
| | magnet device |
| 31 | damping channel |
| 32 | control device |
| 33 | sensor device |
| 34 | direction |
| 35 | direction |
| 36 | spring travel |
| 37 | end position damping |
| 38 | insert device |
| 39 | flow cross section |
| 40 | diameter, length |
| 41 | diameter |
| 42 | lowering valve |
| 43 | magnetic field source, |
| | magnet device |
| 44 | electric coil |
| 45 | remanence magnet |
| 46 | permanent magnet |
| 47 | lowered position |
| 48 | width |
| 49 | distance |
| 50 | spring device |
| 55 | channel |
| 57 | compartment |
| 58 | insulating material |
| 59 | core |
| 60 | ring |
| 61 | field line |
| 62 | valve? |
| 63 | valve? |
| 64 | detector |
| 65 | measuring device |
| 66 | piston surface |
| | compression stage |
| 67 | piston surface rebound |
| | stage |
| 68 | piston rod surface |
| 76 | leg prosthesis |

1-22. (canceled)

23. A damper, comprising:
a damper chamber and a controllable throttle valve formed with at least one damping channel;
a movable piston connected to a piston rod and dividing said damper chamber into a first chamber and a second chamber, wherein said first chamber is connected to said second chamber via a return channel and said throttle valve;
an electronic control device and a magnetic field source controlled by said electronic control device, said magnetic field source being associated with said throttle valve and configured to apply a magnetic field to a magnetorheological damping medium flowing through said at least one damping channel of said throttle valve;
a compensation chamber with a pre-loaded compensation volume connected to said throttle valve and said second chamber;
said throttle valve, said magnetic field source, and said compensation chamber being arranged externally of said damper chamber;
a one-way circuit for conducting the magnetorheological damping medium, the one-way circuit containing at least two one-way valves to enable the damping medium to flow around in the same direction of circulation both when said piston rod plunges into said damper chamber and when said piston rod emerges from said damper chamber, a first of said one-way valves being arranged on said piston and allowing a flow of the damping medium from said second chamber into said first chamber, and a second said one-way valve being disposed between said throttle valve and said second chamber and allowing the damping medium to flow from said throttle valve into said second chamber.

24. The damper according to claim 23, wherein said throttle valve is disposed axially adjacent said damper chamber.

25. The damper according to claim 23, wherein said compensation chamber is disposed axially distanced from said piston.

26. The damper according to claim 25, wherein said throttle valve is connected to said compensation chamber via a first check valve, which allows only a flow of the damping medium from said throttle valve into said compensation chamber, and/or said compensation chamber is connected to said second chamber via a second check valve, which allows only a flow of the damping medium from said compensation chamber into said second chamber.

27. The damper according to claim 23, wherein said throttle valve is connected to said second chamber via a connecting channel.

28. The damper according to claim 23, wherein a ratio of an outer diameter of said piston rod to an outer diameter of said piston lies between 0.2 and 0.4, and/or a ratio of the outer diameter of said piston rod to the outer diameter of said piston is adapted to a predefined ratio of a basic damping in the compression stage and a basic damping in the rebound stage.

29. The damper according to claim 23, wherein said magnetic field source having an electric coil mounted outside said first and second chambers and said coil having a coil axis oriented transversely to a flow direction of the magnetorheological damping medium.

30. The damper according to claim 29, which comprises at least one sensor device disposed for identification of at least one control variable.

31. The damper according to claim 30, wherein said at least one sensor device is configured to detect a measure for a relative speed between said piston and said damper chamber, and/or a sensor device configured to detect a direction of a relative movement between said piston and said damper chamber, and/or a sensor device configured to detect a measure for a spring travel and/or at least an acceleration.

32. The damper according to claim 23, wherein, in intended operation of the damper, said first chamber is arranged below said second chamber and said throttle valve is arranged above said damper chamber.

33. The damper according to claim 32, wherein said compensation chamber is disposed above said throttle valve.

34. The damper according to claim 23, wherein at least said damper chamber and said throttle valve are arranged in a tube system, and wherein an insert device is disposed between said tube system and said damper chamber, and said return channel is formed, at least in portions, at said insert device.

35. The damper according to claim 34, wherein a maximum diameter of a flow cross section of said return channel at said insert device is smaller than a diameter of said tube system.

36. The damper according to claim 34, wherein a mean density of said insert device between said tube system is smaller than a mean density of the damping medium.

37. The damper according to claim 23, wherein a flow of the magnetorheological damping medium is variable at said throttle valve by way of said magnetic field source and a switched state thereof is enabled to be held currentlessly.

38. The damper according to claim 23, which comprises a further throttle valve forming a lowering valve with a further magnetic field source, and wherein said lowering valve is connected in series with said throttle valve, and said further magnetic field source comprises one or more components selected from the group consisting of an electric coil, a remanence magnet, and at least one permanent magnet.

39. The damper according to claim 38, wherein said permanent magnet is movable between a normal position and a lowered position.

40. The damper according to claim 23, wherein a pre-load pressure in said compensation volume lies below 5 bar.

41. The damper according to claim 23, wherein at least one of said one-way valves and/or at least one check valve is a shim valve.

42. In combination with a prosthesis device, the damper according to claim 23.

43. In combination with a motor-driven bike, the damper according to claim 23.

44. In combination with a seat of a vehicle, the damper according to claim 23 for supporting the seat, the vehicle being selected from the group consisting of a passenger car, a truck, a bus, a military vehicle, a utility vehicle, a squad car, a tank, a helicopter, a land vehicle, and a construction machine.
